(19) United States
(12) Patent Application Publication
(10) Pub. No.: US 2023/0151985 A1
(43) Pub. Date: May 18, 2023

(54) AIR-CONDITIONING APPARATUS

(71) Applicant: MITSUBISHI HEAVY INDUSTRIES POWER ENVIRONMENTAL SOLUTIONS, LTD., Yokohama-Shi, Kanagawa (JP)

(72) Inventors: Kazutaka Tomimatsu, Hyogo (JP); Masaya Kato, Kanagawa (JP); Yasutoshi Ueda, Tokyo (JP)

(73) Assignee: MITSUBISHI HEAVY INDUSTRIES POWER ENVIRONMENTAL SOLUTIONS, LTD., Yokohama-Shi, Kanagawa (JP)

(21) Appl. No.: 17/916,688

(22) PCT Filed: Mar. 31, 2021
(86) PCT No.: PCT/JP2021/013777

§ 371 Date: Oct. 3, 2022

(30) Foreign Application Priority Data
Apr. 7, 2020 (JP) 2020-069025

(51) Int. Cl. F24F 8/26; F24F 8/108

(52) U.S. Cl. CPC F24F 8/26; F24F 8/108; F24F 2110/74

(57) ABSTRACT

An air-conditioning apparatus including an electrical precipitator part that has an electric discharge electrode having a body portion and corona discharge portions being for corona discharge and protruding from the body portion, and a collecting electrode that is provided opposite the electric discharge electrode, and an medium-efficiency particulate air filter part that is provided downstream of the electrical precipitator part , wherein the collecting electrode is a plate-shaped member, a plate surface thereof is provided parallel to a gas flow direction, and the corona discharge portion has a first corona discharge section that protrudes from the body portion at one side end of the body portion, upstream in the gas flow direction, and a second corona discharge section that protrudes from the body portion at the other side end of the body portion, downstream in the gas flow direction.

### TECHNICAL FIELD

The present disclosure relates to an air-conditioning apparatus.

### BACKGROUND ART

PM2.5 (fine particulate matter), which is small particles of 2.5 μm or less suspended in the air, has an adverse effect on health, and thus environmental standard values are stipulated in the Basic Environment Law. Although environmental standards are almost observed throughout the year in Japan, various air purification devices have been developed and sold, with health taken into account, so that more purified air is ensured in areas where environmental standards cannot be achieved or in spaces such as rooms of buildings and the like. In addition, abroad, needs for air purification devices are high in areas with poor environmental conditions.

In household air purification devices, HEPA filters capable of removing 99.97% of 0.3 μm particles have been used. With the HEPA filter, fine particulate matter such as PM2.5 having a distribution peak near 0.4 μm can be collected. Meanwhile, also in a commercial air-conditioning apparatus that treats a large amount of air, a filter is provided inside to remove suspended air dust from a space.

However, HEPA filters are not usually used in commercial air-conditioning apparatuses because the filter mesh is fine and clogging easily occurs, and a medium-efficiency particulate air filter, a compact electrostatic filter, or the like is installed as a dust removal device for particle collection. Environmental conditions are satisfied by the above configuration in a case where air conditioning is performed during indoor air recirculation under environmental conditions with little fine particulate matter such as PM2.5.

### CITATION LIST

### Patent Literature

[PTL 1] International Publication No. WO2012/035757

### SUMMARY OF INVENTION

### Technical Problem

When a HEPA filter is applied, energy consumption increases due to the pressure loss thereof. In an air-conditioning apparatus that takes in outside air, such as an air handling unit, HEPA filter application is disadvantageous in terms of energy. Accordingly, in an air-conditioning apparatus that treats a large amount of air using a medium-efficiency particulate air filter instead of a HEPA filter, fine particulate matter such as PM2.5 cannot be sufficiently removed, and thus an air purification device has to be separately installed in a case where a highly air-purified environment is required.

In addition, in the case of indoor air recirculation by an air conditioning system, fine particulate matter (submicron particle) generated indoors, a virus emitted from a person, or the like is hardly collected by a filter installed in the air-conditioning apparatus. As a result, these substances continue to remain indoors.

It should be noted that PTL 1 discloses a technique in which the inside of the main body of the indoor unit of an air conditioner is provided with an ozone-ion generator for ozone and ionic wind generation by discharge so that the inside of the main body is purified and sterilization is performed by ozone diffusion.

The present disclosure has been made in view of such circumstances, and an object of the present disclosure is to provide an air-conditioning apparatus that is capable of improving the efficiency of collection of fine particles in particular without an increase in pressure loss and performing deodorization or sterilization with respect to intra-space air.

### Solution to Problem

In order to solve the above problems, the air-conditioning apparatus of the present disclosure adopts the following means.

In other words, an air-conditioning apparatus according to the present disclosure includes: an electrical precipitator unit including a discharge electrode having a main body portion and a corona discharge portion for corona discharge protruding from the main body portion and a collecting electrode installed to face the discharge electrode; and a medium-efficiency particulate air filter unit installed downstream of the electrical precipitator unit, in which the collecting electrode is a plate-shaped member and has a plate surface provided parallel to a gas flow direction, and the corona discharge portion has a first corona discharge portion protruding from the main body portion toward an upstream side in the gas flow direction in one side end portion of the main body portion and a second corona discharge portion protruding from the main body portion toward a downstream side in the gas flow direction in the other side end portion of the main body portion.

### Advantageous Effects of Invention

According to the present disclosure, it is possible to improve the efficiency of collection of fine particles in particular without an increase in pressure loss and perform deodorization or sterilization with respect to intra-space air.

### BRIEF DESCRIPTION OF DRAWINGS

FIG. 1 is a configuration diagram illustrating an air-conditioning apparatus according to an embodiment of the present disclosure.

FIG. 2 is a configuration diagram illustrating an air handling unit of an air-conditioning apparatus according to an embodiment of the present disclosure.

FIG. 3 is a configuration diagram illustrating a modification example of an air handling unit of an air-conditioning apparatus according to an embodiment of the present disclosure.

FIG. 4 is a lateral cross-sectional view illustrating an electrical precipitator unit and a medium-efficiency particulate air filter unit of an air handling unit according to an embodiment of the present disclosure.

FIG. 5 is a vertical cross-sectional view illustrating an electrical precipitator unit of an air handling unit according to an embodiment of the present disclosure.

FIG. 6 is a perspective view illustrating an electrical precipitator unit of an air handling unit according to an embodiment of the present disclosure.

FIG. 7 is a timing chart illustrating an example of an operation of an air handling unit of an air-conditioning apparatus according to an embodiment of the present disclosure.

FIG. 8 is a timing chart illustrating an example of an operation of a continuous energization condition of an air-conditioning apparatus according to an embodiment of the present disclosure.

FIG. 9 is a timing chart illustrating an example of an operation of an intermittent energization condition of an air-conditioning apparatus according to an embodiment of the present disclosure.

FIG. 10 is a graph illustrating a relationship between ozone concentration or submicron particle collection efficiency and electric power by an air-conditioning apparatus according to an embodiment of the present disclosure.

FIG. 11 is a timing chart illustrating an example of an operation of a fan coil unit of an air-conditioning apparatus according to an embodiment of the present disclosure.

### DESCRIPTION OF EMBODIMENTS

Hereinafter, an air-conditioning apparatus 1 according to an embodiment of the present disclosure will be described with reference to the drawings.

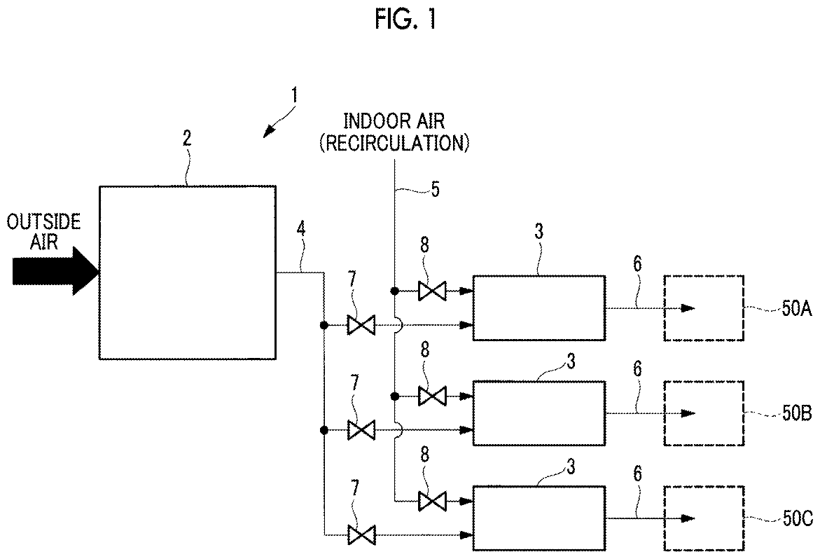

The air-conditioning apparatus 1 according to the present embodiment takes in external air (outside air) such as the atmosphere, performs temperature or humidity adjustment, and supplies the adjusted air to each space 50 provided in a building. As illustrated in FIG. 1, the air-conditioning apparatus 1 includes an outside air treatment air conditioner (hereinafter, referred to as “external air conditioner”) 2, a plurality of air handling units (hereinafter, referred to as “AHUs”) 3, ducts 4, 5, and 6, dampers 7 and 8, and so on.

The duct 4 is installed between the external air conditioner 2 and the AHU 3, one end thereof is connected to the external air conditioner 2, and the other end thereof is connected to the outside air intake port of the AHU 3. The duct 5 is installed between each space 50 (50A, 50B, and 50C in the example illustrated in FIG. 1) such as a room of a building or the like and the AHU 3, one end thereof is connected to the suction port provided in each of the spaces 50A, 50B, and 50C, and the other end thereof is connected to the recirculation air intake port of the AHU 3. The duct 6 is installed between the AHU 3 and each of the spaces 50A, 50B, and 50C, one end thereof is connected to the air discharge port of the AHU 3, and the other end thereof is connected to the outlet provided in each of the spaces 50A, 50B, and 50C.

The external air conditioner 2 takes in outside air, performs temperature and/or humidity adjustment of the outside air, and supplies the adjusted air to the AHU 3 via the duct 4. A filter, a heat exchanger, a humidifier, and so on are installed inside the casing of the external air conditioner 2.

The air supplied from the external air conditioner 2 to the plurality of AHUs 3 is branched by the duct 4 and supplied to each AHU 3. As for the duct 4, the damper 7 is installed upstream of the outside air intake port of each AHU 3, and the damper 7 adjusts the amount of outside air supplied to the AHU 3.

The AHU 3 takes in the air supplied from the external air conditioner 2 and the air from the space 50, performs temperature and/or humidity adjustment of the intake air, and supplies the adjusted air to the space 50 via the duct 6. An electrical precipitator unit 10, a medium-efficiency particulate air filter unit 12, an air conditioning unit 13, and so on are installed inside a casing 9 of the AHU 3.

The air is taken into the AHU 3 from the space 50, and the AHU 3 readjusts the temperature and/or humidity of the air. In this manner, in the AHU 3, not only the outside air is taken in and supplied to the space 50, but also the air from the space 50 is recirculated, and energy efficiency can be enhanced as a result. As for the duct 5, the damper 8 is installed upstream of the recirculation air intake port of each AHU 3, and the damper 8 adjusts the amount of recirculation air supplied to the AHU 3.

In the air-conditioning apparatus 1 according to the present embodiment, first, the external air conditioner 2 takes in outside air, the external air conditioner 2 performs temperature and/or humidity adjustment of the outside air, and the air adjusted by the external air conditioner 2 is supplied to the AHU 3. Then, the AHU 3 takes in the air supplied from the external air conditioner 2 and the air from the space 50, the AHU 3 performs temperature and/or humidity adjustment of the intake air, and the air adjusted by the AHU 3 is supplied to the space 50. At this time, the amount of outside air taken into the AHU 3 from the external air conditioner 2 and the amount of recirculation air taken into the AHU 3 from the space 50 are adjusted by the dampers 7 and 8, respectively.

For example, in a cooling or heating season, the amount of outside air taken into the space 50 as fresh air is set to a relatively low ratio (for example, 30%) with respect to the total intake air amount. In addition, in the case of air-conditioning apparatus designed with energy saving taken into account, 100% of outside air may be taken in an intermediate season(spring or autumn), and adjustment is performed such that energy efficiency is enhanced by outside air intake being performed in accordance with the season.

Next, the AHU 3 according to the present embodiment will be described.

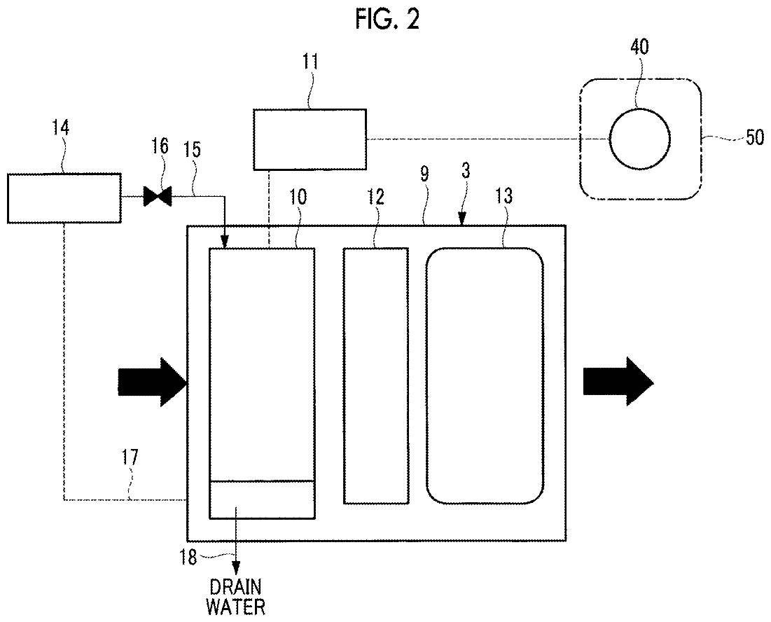

As illustrated in FIG. 2, the AHU 3 has, for example, the electrical precipitator unit 10, a control unit 11, the medium-efficiency particulate air filter unit 12, the air conditioning unit 13, and so on. The electrical precipitator unit 10, the medium-efficiency particulate air filter unit 12, and the air conditioning unit 13 are installed inside the casing 9 of the AHU 3, and the air taken into the AHU 3 flows in the order of the electrical precipitator unit 10, the medium-efficiency particulate air filter unit 12, and the air conditioning unit 13. The treatment speed in the AHU 3 is, for example, in the range of 2.5 m/s to 3.5 m/s applied in a normal AHU.

The electrical precipitator unit 10 removes dust (including particulate matter) contained in the air taken in by the air-conditioning apparatus 1. The electrical precipitator unit 10 includes discharge electrodes 31 charging particles, collecting electrodes 32 installed so as to face the discharge electrode 31, and so on. By corona discharge occurring at the discharge electrode 31, gas molecules are ionized, and the particles contained in the air are energized as the particles pass through the electric field between the electrodes. Then, the energized particles are attached at the collecting electrode 32 and collected.

The control unit 11 adjusts the voltage or energization condition applied to the electrical precipitator unit 10. The control unit 11 receives a signal related to measurement data from an ozone concentration measuring unit 40. In addition, the control unit 11 transmits a control signal for voltage or energization condition adjustment to the electrical precipitator unit 10.

The medium-efficiency particulate air filter unit 12 is installed downstream of the electrical precipitator unit 10 and removes dust contained in the air that has passed through the electrical precipitator unit 10. A medium-efficiency particulate air filter 33 normally used for the AHU 3 can be applied to the medium-efficiency particulate air filter unit 12. The medium-efficiency particulate air filter 33 is, for example, a sheet member and has a pleated multi-fold structure.

It should be noted that the medium-efficiency particulate air filter applied as the medium-efficiency particulate air filter 33 in the present embodiment is defined in JIS terminology as having medium degree particle collection efficiency mainly with respect to small particles of 5 μm or less. In addition, according to general literature as to the performance of a medium-efficiency particulate air filter, the efficiency of collection of particles with a medium diameter of 1.6 μm to 2.3 μm is approximately 50% to 80% by the so-called colorimetric method and the efficiency of collection by the DOP method (0.3 μm particle) is approximately 15% to 50%. It should be noted that the inventor has found regarding the medium-efficiency particulate air filter that in an experiment using atmospheric dust that does not show characteristic of attachment as in the case of DOP particles, even 0.4 μm particles show a collectability of as low as approximately 15% to 25% and most fine submicron particles slip through.

The air conditioning unit 13 performs temperature and/or humidity adjustment of the air that has passed through the electrical precipitator unit 10 and the medium-efficiency particulate air filter unit 12 and supplies the adjusted air to the space 50. The air conditioning unit 13 has a heat exchanger, a humidifier, and so on.

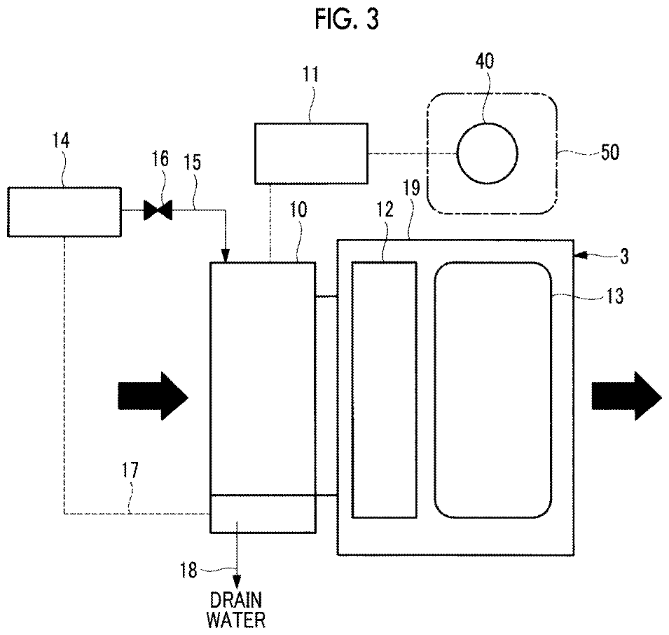

It should be noted that a case where the AHU 3 described above is an integrated AHU in which the electrical precipitator unit 10 is installed inside the casing 9 has been described and yet the present disclosure is not limited to this example. As illustrated in FIG. 3, in an example of the AHU 3, the medium-efficiency particulate air filter unit 12 and the air conditioning unit 13 may be installed inside a casing 9 and the electrical precipitator unit 10 may be attached to the outside of the casing 9. In other words, the air handling unit includes one in which the electrical precipitator unit 10 is externally attached with respect to a configuration packaged without incorporating the electrical precipitator unit 10. Accordingly, the air-conditioning apparatus according to the present disclosure can be applied to both the newly installed AHU 3 incorporating the electrical precipitator unit 10 and the AHU 3 at which the electrical precipitator unit 10 is additionally installed.

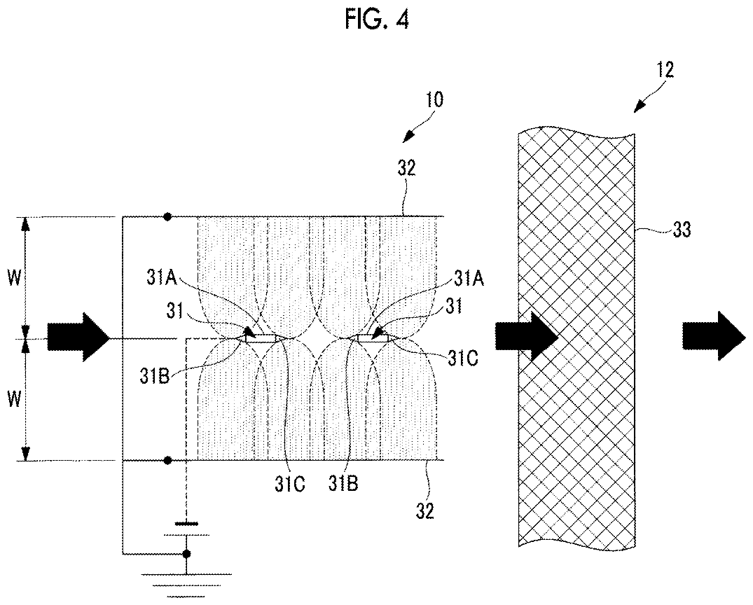

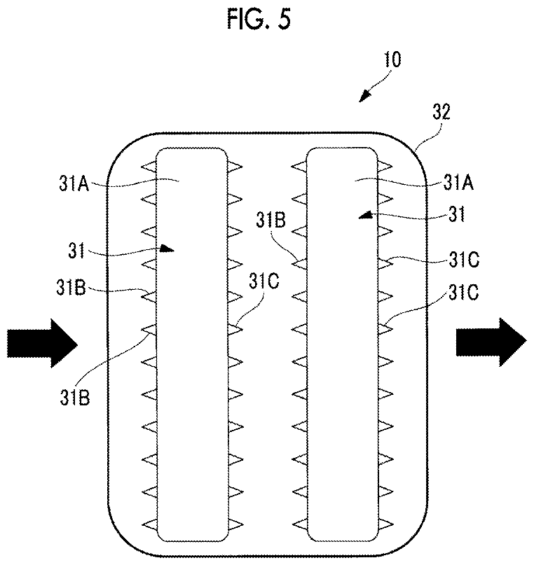

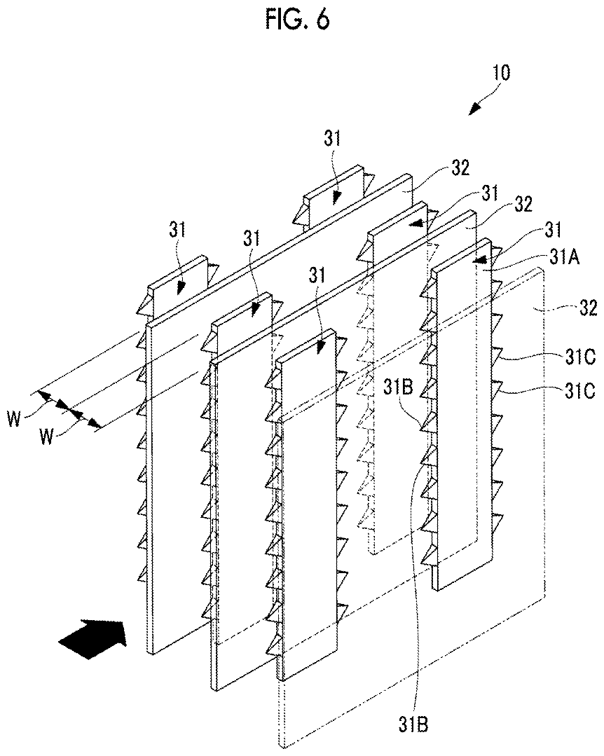

Next, the electrical precipitator unit 10 of the AHU 3 according to the present embodiment will be described with reference to FIGS. 4 to 6.

In the electrical precipitator unit 10, gas flows in one direction from the upstream side to the downstream side of the AHU 3.

The collecting electrodes 32, which are, for example, metallic plate-shaped members, are installed in the electrical precipitator unit 10. The collecting electrode 32 has a plate surface provided parallel to the gas flow direction. The plurality of collecting electrodes 32 are installed at predetermined intervals in a direction orthogonal to the gas flow direction. The collecting electrode 32 is, for example, an opening portion-less flat plate-shaped member, a reticulated member having an opening portion, a punching metal, or the like.

The discharge electrode 31 is installed between the collecting electrodes 32 that are adjacent to each other. The discharge electrode 31 has a main body portion 31A and corona discharge portions 31B and 31C, and the corona discharge portions 31B and 31C are provided so as to protrude from the main body portion 31A. The corona discharge portions 31B and 31C have, for example, a spiny shape.

At least one discharge electrode 31 may be provided, and the corona discharge portions are two or more stages in total. In the example illustrated in FIGS. 4 to 6, two discharge electrodes 31 are installed along the gas flow direction. The main body portion 31A of the discharge electrode 31 is a long plate-shaped member that is long in one direction. It should be noted that the plate surface of the main body portion 31A may be provided with, for example, circular openings (through-holes) at predetermined intervals along the length direction and the main body portion 31A may be an opening-less flat plate.

The main body portion 31A has a plate surface provided parallel to the gas flow direction. The main body portion 31A is installed such that the length direction of the main body portion 31A is orthogonal to the gas flow direction and orthogonal to the direction in which the plurality of collecting electrodes 32 are installed.

The corona discharge portion 31B protrudes toward the upstream side in the gas flow direction in one side end portion of the main body portion 31A, for example, the upstream side end portion in the gas flow direction. The corona discharge portion 31B is an example of a first corona discharge portion. In addition, the corona discharge portion 31C protrudes toward the downstream side in the gas flow direction in the other side end portion of the main body portion 31A, for example, the downstream side end portion in the gas flow direction. The corona discharge portion 31C is an example of a second corona discharge portion.

Corona discharge occurs at the corona discharge portions 31B and 31C, and ionic wind is generated from the tips of the corona discharge portions 31B and 31C toward the facing collecting electrode 32 side. In other words, the discharge electrode 31 can be subjected to corona discharge from the corona discharge portions 31B and 31C toward the collecting electrode 32 to allow ionic wind to flow.

In addition, in each discharge electrode 31, a total of two stages of corona discharge portions are provided with the corona discharge portion 31B provided on the upstream side and the corona discharge portion 31C provided on the downstream side. Further, for example, as illustrated in FIGS. 4 to 6, a total of four stages of corona discharge portions are provided in a case where two discharge electrodes 31 are provided in the electrical precipitator unit 10.

An interval W between the surface of the discharge electrode 31 and the surface of the collecting electrode 32 is set in the range of, for example, 10 mm or more and 40 mm or less. In a general electrical precipitator, discharge and collecting electrodes have an interval in the range of 150 mm or more and 250 mm or less. In other words, the interval W between the discharge electrode 31 and the collecting electrode 32 is relatively narrow. In a case where the interval W between the discharge electrode 31 and the collecting electrode 32 is narrow, the collecting area per unit amount can be increased. However, at the interval W that is too small, the dust collected by the collecting electrode 32 may cause local electric field concentration. Accordingly, the interval W that is ensured is preferably 10 mm or more.

In the present embodiment, collection performance improvement is achieved since the corona discharge portions 31B and 31C are provided in a plurality of stages. In existing air purification devices, corona current is suppressed as much as possible for ozone generation suppression. In addition, of the existing air purification devices, an electrical precipitator is configured based on dust charging in a charging portion and collection by Coulomb force under the electric field on the downstream side of the charging portion regarding dust collection. Of the existing air purification devices, an electrostatic filter is configured based on dust charging in a charging portion and collection by Coulomb force acting by the energization of the particles in a filter on the downstream side of the charging portion regarding dust collection. Accordingly, in each case, the charging portion is provided at only one place from the viewpoint of suppressing ozone generation and from the viewpoint that collection by Coulomb force on the downstream side of the charging portion being possible is better.

On the other hand, in the electrical precipitator unit 10 according to the present embodiment, corona discharge is performed more stably than in the case of a positive charge by negative charge application to the discharge electrode 31. In addition, the electrical precipitator unit 10 is configured based on dust charging and collection by corona current continuation regarding dust collection, and collection is performed inside the electrical precipitator unit 10 as well. In the electrical precipitator unit 10, ionic wind is sustained by dust being energized and corona current being ensured, and the ionic wind also promotes dust collection. Collection using ionic wind is also realized since the corona discharge portions 31B and 31C are provided in a plurality of stages along the gas flow direction.

In addition, the electrical precipitator unit 10 is configured to actively generate ozone. With the generated ozone, it is possible to deodorize the air in the space 50, inactivate a virus contained in the air, and sterilize fungi. As for existing air purification devices, ozone generation suppression is a task. On the other hand, in the present embodiment, the amount of ozone generation is adjusted by adjusting the voltage or energization condition applied to the discharge electrode 31 of the electrical precipitator unit 10 based on ozone concentration and environmental conditions.

The medium-efficiency particulate air filter 33 has a low pressure loss and a large dust holding capacity. In addition, the electrical precipitator unit 10 is installed upstream of the medium-efficiency particulate air filter unit 12, dust is collected at the electrical precipitator unit 10 as well, and thus the amount of dust collected by the medium-efficiency particulate air filter 33 and the frequency of replacement of the medium-efficiency particulate air filter 33 are reduced. In addition, at the electrical precipitator unit 10 on the upstream side, a sufficient amount of energization can be applied to particles in a diffusion charging process (for example, submicron particles) by the plurality of stages of corona discharge portions 31B and 31C, and thus a strong electrostatic force acts on the main body of the medium-efficiency particulate air filter 33. As a result, the collection efficiency of the medium-efficiency particulate air filter unit 12, fine particle collection efficiency in particular, is significantly improved.

The discharge electrode 31 is connected to a power supply having a negative polarity, and the collecting electrode 32 is grounded and has a positive polarity. Stable discharge is possible in a case where a negative charge is applied to the discharge electrode 31. In addition, it is easy to generate ozone at the time of discharge by applying a negative charge to the discharge electrode 31. It should be noted that the present disclosure is not limited to this example, a positive charge may be applied to the discharge electrode 31, and the collecting electrode 32 may be used as a negative electrode.

Next, the control of the electrical precipitator unit 10 according to the present embodiment will be described.

The ozone concentration measuring unit 40 is installed in the space 50, and air is supplied from the AHU 3 to the space 50 after temperature and/or humidity adjustment, that is, passage through the electrical precipitator unit 10 and the medium-efficiency particulate air filter unit 12. The ozone concentration measuring unit 40 measures the ozone concentration in the space. Data related to the measured ozone concentration is transmitted from the ozone concentration measuring unit 40 to the control unit 11.

The control unit 11 adjusts the voltage or energization condition applied to the discharge electrode 31 of the electrical precipitator unit 10 based on the measured ozone concentration. As a result, the amount of ozone generated as a result of the corona discharge at the discharge electrode 31 is adjusted. In a case where the ozone concentration in the space 50 is increased, the control unit 11 changes the voltage or energization condition applied to the discharge electrode 31 of the electrical precipitator unit 10 to increase the input power. On the other hand, in a case where the ozone concentration in the space 50 is lowered, the control unit 11 changes the voltage or energization condition applied to the discharge electrode 31 of the electrical precipitator unit 10 to reduce the input power. Alternatively, the energization is paused as needed.

The control unit 11 applies a continuous energization condition or an intermittent energization condition in a case where the control unit 11 adjusts the energization condition applied to the discharge electrode 31 of the electrical precipitator unit 10.

As for the continuous energization condition, full-wave rectification is performed in a direct-current high-voltage power supply device (transformer rectifier), and direct current is applied to the discharge electrode 31. Voltage level adjustment is performed by an increase or decrease in supply current on the primary side. The current attributable to corona discharge and flowing through the electrical precipitator unit 10 also increases or decreases in accordance with the voltage level, the amount of ozone generation changes, and the ozone concentration changes. It should be noted that it is possible to switch between energization ON and OFF in the case of the continuous energization condition as well as the intermittent energization to be described later. The ON-OFF switching of the continuous energization condition is controlled by, for example, an external timer and is on the order of at least a few seconds.

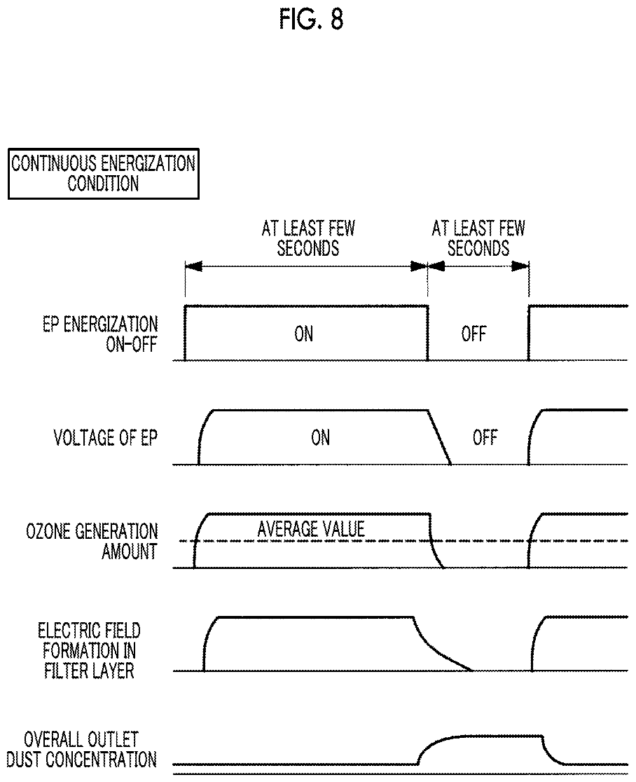

In the continuous energization condition, as illustrated in FIG. 8, the amount of ozone generation increases as the voltage and current increase by the energization of the electrical precipitator unit 10 being turned ON and the amount of ozone generation decreases as the voltage and current decrease by the energization of the electrical precipitator unit 10 being turned OFF. With the energization of the electrical precipitator unit 10 OFF, the dust passing through the electrical precipitator unit 10 is not energized, and thus no energized dust flies to the medium-efficiency particulate air filter unit 12 on the downstream side. As a result, at the time of energization OFF, no electric field is formed in the medium-efficiency particulate air filter 33, no energization can be maintained, and thus the dust concentration at the outlet on the downstream side of the medium-efficiency particulate air filter unit 12 tends to increase.

On the other hand, as for the intermittent energization condition, in a commercial frequency-based direct-current high-voltage power supply device (transformer rectifier), the output on the primary side of the transformer is intermittently turned OFF. For example, the energizing rate is reduced to 1/3 by turning ON (adopting) one out of three mountains and turning OFF the other two. As for energization at this time, the energization is turned ON and OFF in units of 10 milliseconds in an area of, for example, 50 Hz, and thus the ON timing is every 30 milliseconds and ON and OFF are repeated in a case where the energizing rate is ⅓. In addition, in the case of a high-frequency power supply or energizing condition by a boosting voltage method using an electronic circuit, control in finer frequency units is possible and, in that case, control for turning ON the energization every 1 to 3 milliseconds and repeating ON and OFF is also possible. Then, the amount of ozone generation changes in accordance with the energizing rate, and the ozone concentration changes.

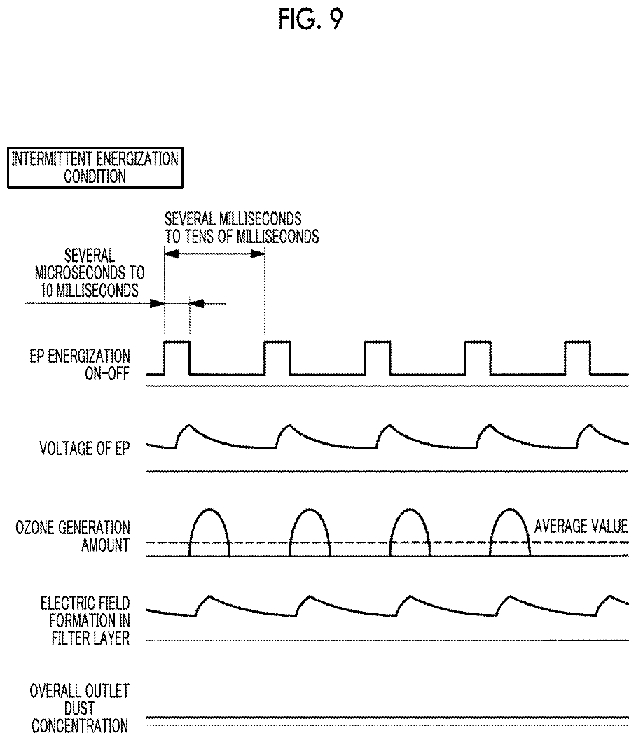

In the intermittent energization condition, as illustrated in FIG. 9, with the energization of the electrical precipitator unit 10 ON, charging current flows through the capacitor component of the electrical precipitator unit 10 to lead to a rise in voltage and current flows as a result of corona discharge, and then discharge current flows and the voltage gradually decreases in the duration of an OFF state where no new energization is turned ON. Also in the intermittent energization condition, the amount of ozone generation increases as the voltage and current increase by the energization of the electrical precipitator unit 10 being turned ON and the amount of ozone generation decreases as the voltage and current decrease by the energization of the electrical precipitator unit 10 being turned OFF. Even with the energization of the electrical precipitator unit 10 OFF once, the cycle is short in the intermittent energization condition, and thus the energization is turned ON again during dust passage through the electrical precipitator unit 10 as well and, as a result, energization is turned ON and OFF many times and the dust itself is always energized. Accordingly, energized dust always flies to the medium-efficiency particulate air filter unit 12 on the downstream side. As a result, a certain amount of energization is held in the medium-efficiency particulate air filter 33, and thus a situation is maintained in which the dust concentration at the outlet on the downstream side of the medium-efficiency particulate air filter unit 12 is stably reduced.

By the intermittent energization condition as compared with the continuous energization condition, electric power can be reduced, energy can be saved, and high filter performance can be maintained by the electric field in the medium-efficiency particulate air filter unit 12 being maintained. In addition, the input power can be suppressed to a low level, and thus the ozone concentration can also be suppressed to a low level.

The control unit 11 switches between a first mode in which the voltage or energization condition applied to the discharge electrode 31 of the electrical precipitator unit 10 is adjusted such that the measured ozone concentration becomes equal to or greater than a predetermined threshold value and a second mode in which the voltage or energization condition applied to the discharge electrode 31 of the electrical precipitator unit 10 is adjusted such that the measured ozone concentration becomes less than the predetermined threshold value.

The control unit 11 is configured by, for example, a central processing unit (CPU), a random access memory (RAM), a read-only memory (ROM), a computer-readable storage medium, and so on. A series of processing for realizing various functions is, for example, stored in the storage medium or the like in the form of a program, the

CPU reads this program into the RAM or the like to execute information and/or arithmetic processing, and the various functions are realized as a result. It should be noted that the program may be applied in the form of being, for example, pre-installed in the ROM or another storage medium, provided in a state of being stored in the computer-readable storage medium, or distributed via wired or wireless communication means. The computer-readable storage medium is a magnetic disk, a magneto-optical disk, a CD-ROM, a DVD-ROM, a semiconductor memory, or the like.

The predetermined threshold value is, for example, set to the extent that the ozone concentration in the space 50 is sufficiently below an environmental standard value (0.1 ppm).

For example, in the first mode, the ozone concentration is increased such that the space supplied with air is forcibly deodorized or sterilized (including virus inactivation) by ozone and, in the second mode, the ozone concentration is lowered to the extent that ozone does not adversely affect a person staying in the space.

It should be noted that the ozone concentration in the space 50 may be adjusted based on the voltage applied to the discharge electrode 31 and environmental conditions such as the temperature and humidity in the space 50. In general, ozone concentration changes depending on the temperature or humidity in a space. Pre-acquired in this regard is a correlation such as a characteristic curve related to the relationship between the temperature and/or humidity in the space 50 and the ozone concentration at the time when any voltage is applied to the discharge electrode 31. Then, based on the measured temperature and/or humidity and the characteristic curve, the voltage or energization condition applied to the discharge electrode 31 is adjusted such that a desired ozone concentration is reached. In this case, the temperature and/or humidity in the space 50 is measured with a thermometer and/or hygrometer installed at the AHU 3 or the like, and the measurement result is transmitted to the control unit 11. Here, each of the thermometer and the hygrometer is an example of an environmental condition measuring unit.

In addition, in a case where the correlation is acquired in advance, only the temperature and/or humidity in the space 50 may be measured without installing the ozone concentration measuring unit 40 in the space 50. In this case, the voltage or energization condition applied to the discharge electrode 31 of the electrical precipitator unit 10 is adjusted based on the relationship between the voltage applied to the discharge electrode 31 and the measured environmental conditions.

As described above, the adjustment of the ozone concentration in the space 50 may be performed by feedback control based on the measurement result of the ozone concentration measuring unit 40 and/or an environmental condition measurement result such as temperature and humidity measurement results or may be performed by switching between the first mode of high concentration and the second mode of low concentration in accordance with a time slot.

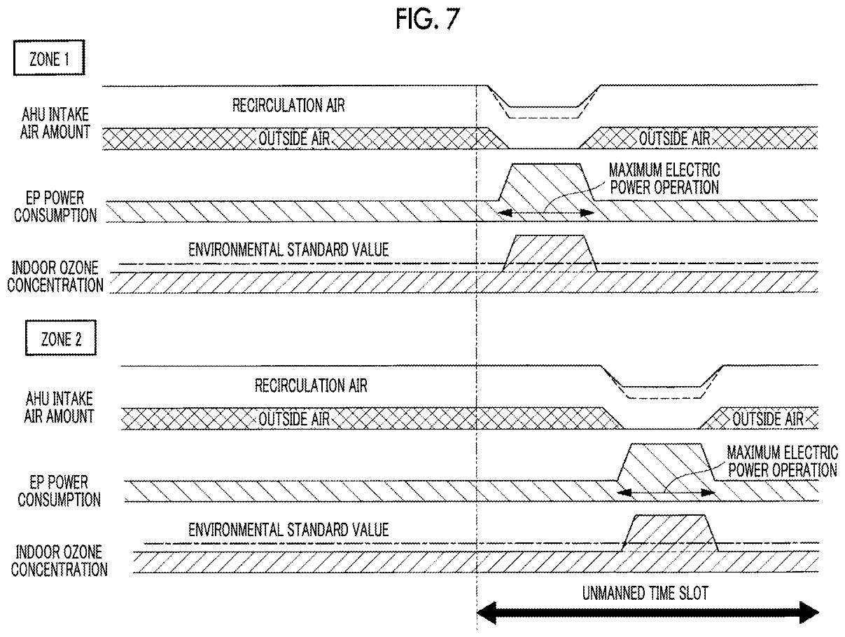

For example, as illustrated in FIG. 7, in a time slot when a person stays in the space 50, a switch to the second mode is performed with the air-conditioning apparatus 1 in operation and the voltage applied to the discharge electrode 31 is reduced such that the ozone concentration becomes equal to or less than the environmental standard value. As a result, the ozone concentration is reduced to the extent that ozone does not adversely affect the person staying in the space. On the other hand, when no one stays in the space 50 or in a state where no one is allowed to enter the space 50, a switch to the first mode is performed and the voltage applied to the discharge electrode 31 is increased such that the ozone concentration becomes a high value. As a result, the ozone concentration is increased such that the space supplied with air is forcibly deodorized or sterilized by ozone.

The voltage applied to the discharge electrode 31 in the second mode is lower than the voltage applied to the discharge electrode 31 in the first mode operated so as to be forcibly deodorized or sterilized by ozone. However, the voltage value of the second mode is set such that dust can be efficiently collected by the electrical precipitator unit 10 even at the voltage applied to the discharge electrode 31 in the second mode. In addition, the voltage applied in the first mode is, for example, the maximum value that can be applied by the electrical precipitator unit 10. As a result, the amount of ozone generated at the electrical precipitator unit 10 can be maximized and the ozone concentration can be increased rapidly.

When a switch to the first mode is performed in a time slot when no one stays in the space 50 or in a state where no one is allowed to enter the space 50, the outside air intake at the AHU 3 may be stopped and the ratio of the amount of recirculation air to the total intake air amount of the AHU 3 may be caused to reach 100% (see the AHU intake amount in FIG. 7) so that the ozone concentration is efficiently increased. In addition, when the outside air intake at the AHU 3 is stopped after a switch to the first mode, the amount of recirculation air may be reduced more than the amount of recirculation air in the second mode (see the broken line portion of the AHU intake amount in FIG. 7). As a result, the ozone concentration of the air supplied from the AHU 3 to the space 50 can be increased.

Further, a predetermined CT value may be ensurable so that the effect of sterilization or deodorization in the space 50 is obtained. The CT value is a value (ppm·min) expressed by the product of the ozone concentration (ppm) and the time (min) of contact with an object to be treated at the ozone concentration. Accordingly, even in a case where the ozone concentration is low, by setting a long contact time, it is possible to ensure a CT value equivalent to that in the case of contact with a high ozone concentration in a short time. For example, the first mode may be set to a relatively long time in a case where the absolute ozone concentration is suppressed or in a case where there is a limit to the amount of ozone generated at the electrical precipitator unit 10.

In an office building or a large space where people cannot enter depending on the time slot (for example, a theater), it is also possible to automatically switch between the first mode and the second mode using an unmanned time slot at night.

In addition, in the case of returning from a state where the ozone concentration in the space 50 is high to a state where the ozone concentration is low and satisfies the environmental standard value, a switch is performed from the first mode of high concentration to the second mode of low concentration. In addition, at this time, the return can be expedited by temporarily increasing the amount of outside air taken into the AHU 3 as compared with the normal operation.

In a case where the plurality of AHUs 3 are installed and the AHUs 3 supply air to different spaces 50, as illustrated in FIG. 7, a switch to the first mode is performed for each space 50 and a target space is deodorized or sterilized. For example, in a case where deodorization or sterilization is performed in one space 50 (Zone 1 in the example illustrated in FIG. 7), only the target space is set to the first mode and the other space 50 (Zone 2 in the example illustrated in FIG. 7) remains in the second mode.

In the space 50 set to the first mode, the flow of outside air into the space 50 is blocked, the amount of recirculation air is set to 100%, and the voltage applied to the discharge electrode 31 in the electrical precipitator unit 10 is set to, for example, the maximum value. As a result, the target space is deodorized or sterilized.

At this time, the other space 50 may remain in normal operation in a case where the recirculation air line that connects the AHU 3 and the space 50 is independent for each space 50. On the other hand, in a case where the recirculation air line is common and the air in the plurality of spaces 50 is suctioned in and returned to the AHU 3, the amount of recirculation air in the target space is increased and the amount of recirculation air in the other space 50 is decreased. As a result, the amount of recirculation air in the target space supplied from the AHU 3 increases, and thus the ozone concentration can be efficiently increased.

In addition, a washing liquid supply unit 14 for washing the electrical precipitator unit 10 may be installed at the AHU 3 as illustrated in FIGS. 2 and 3. A liquid such as hypochlorite water, ozone water and so on is supplied from the washing liquid supply unit 14 to the collecting electrode 32 via a supply pipe 15, and the liquid flows on the surface of the collecting electrode 32. A valve 16 is installed on the supply pipe 15, and the valve 16 controls the start and stop of the supply of the liquid supplied to the collecting electrode 32. As a result, it is possible to wash the dust attached on the surface of the collecting electrode 32 while sterilizing the collecting electrode 32. The liquid that has flowed on the surface of the collecting electrode 32 is discharged to the outside of the AHU 3 via a drain pipe 18 as a drain. It should be noted that in the AHU 3 that is large or the like, the liquid that has flowed on the surface of the collecting electrode 32 may be recovered and the recovered liquid may be returned to the washing liquid supply unit 14 via a recirculation pipe 17 to be reused.

It should be noted that the electrical precipitator unit 10, the medium-efficiency particulate air filter unit 12, and so on can be sterilized in the first mode in which ozone is maintained at a high concentration.

According to the air-conditioning apparatus 1 according to the present embodiment, the electrical precipitator unit 10 and the medium-efficiency particulate air filter unit 12 are provided upstream of the air conditioning unit 13, and collection efficiency improvement can be achieved without an increase in pressure loss unlike in a case where a HEPA filter is installed. The present embodiment is particularly suitable in the case of adoption in an apparatus where it is difficult to adopt a HEPA filter and the amount of treatment air is large. By installing the electrical precipitator unit 10, dust can be collected by the electrical precipitator unit 10 and the medium-efficiency particulate air filter unit 12 is capable of collecting the dust energized by passing through the electrical precipitator unit 10.

The inventor has confirmed that the efficiency of collection of fine particles (submicron particles), viruses, and the like hardly collectible with an existing medium-efficiency particulate air filter can be increased to at least 95% as a result. In addition, since the pressure loss does not increase, energy consumption attributable to power can be reduced as compared with a case where a HEPA filter is installed. It should be noted that here, the efficiency of collection of fine particles (submicron particles), viruses, and the like is in accordance with a mask application reference in the medical field. The collection efficiency of a mask in the medical field is set to 95% by the DOP method (0.3 μm particle) in the application reference. Although the actual collection efficiency of HEPA filter-equivalent masks is 99.97%, due to breathing difficulties, masks in the medical field have been served with the efficiency of collection of viruses and the like set to be equivalent to 95%. Even at the 95% collection efficiency confirmed in the present embodiment, practical use is possible from the viewpoint of virus removal.

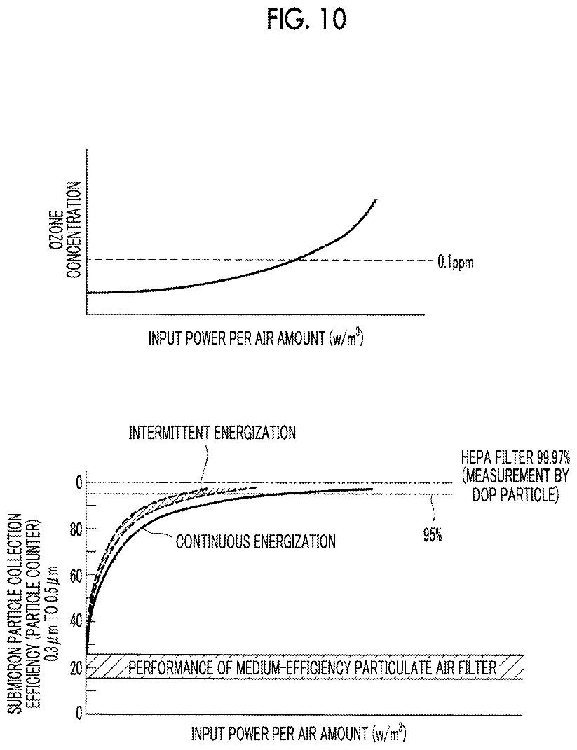

The following findings have been obtained by the inventors. In other words, as for the electrical precipitator unit 10, when the input power per air amount is increased, the collection efficiency of the whole including the electrical precipitator unit 10 and the medium-efficiency particulate air filter unit 12 is improved as illustrated in FIG. 10. This is because the amount of submicron particle energization also increases and thus the performance of the medium-efficiency particulate air filter unit 12 as well as the electrical precipitator unit 10 is improved to increase the total efficiency.

However, with the input power increased, the ozone concentration also increases, and thus it is desirable to adopt energization condition such as electric power suppression or energizing rate reduction by the intermittent energization condition for an operation at below the environmental standard value (0.1 ppm).

In the present embodiment, the total efficiency of submicron particles significantly rises, as compared with the medium-efficiency particulate air filter unit 12 alone, as a result of the combination with the electrical precipitator unit 10. However, from the viewpoint of virus removal from the space or the like, it is desirable that the exhibited performance is equivalent to 95% or more of aerosol particles adopted in a medical mask. In order to meet this requirement, in the present embodiment, the input power per air amount is changeable such that it is possible to collect 95% or more of 0.3 μm particles of the same size as aerosol particles while maintaining the ozone concentration. As a result, it is possible to reduce ozone concentration and ensure aerosol particle collection efficiency at the same time by increasing or decreasing the current and voltage in the continuous energization condition or changing the energizing rate in the intermittent energization condition.

It should be noted that the air-conditioning apparatus according to the present embodiment may include a fan coil unit (hereinafter, referred to as “FCU”) or the like. In other words, an FCU may be installed instead of the AHU 3 in the first embodiment. As in the case of the AHU 3, the FCU has, for example, the electrical precipitator unit 10, the control unit 11, the medium-efficiency particulate air filter unit 12, the air conditioning unit 13, and so on. In addition, application is possible to both a newly installed FCU incorporating the electrical precipitator unit 10 and an FCU where the electrical precipitator unit 10 is additionally installed. In addition, using an FCU capable of supplying a large amount of air, it is possible to perform air purification by deodorization or sterilization with respect to a large space. For an increase in ozone concentration, it is desirable to maximize the input power of the electrical precipitator unit 10 to maximize the amount of ozone generation and operate an FCU with the amount of treatment air reduced.

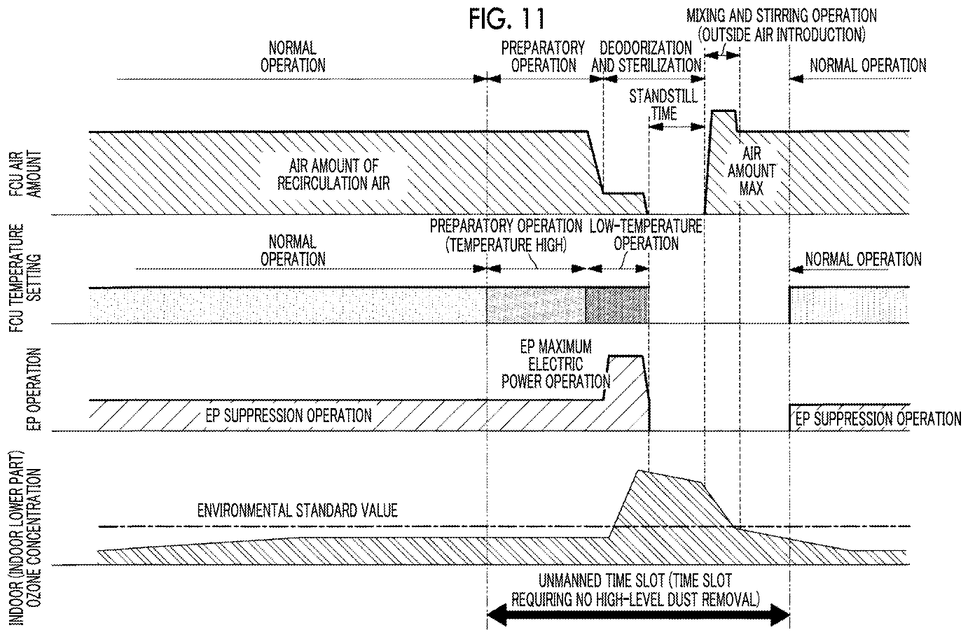

Next, a control method for intensively supplying high-concentration ozone to a human action area will be described with reference to FIG. 11. Here, the human action area is, for example, the space of 2 m height or less from a floor surface. By deodorization or sterilization within the range of this space, adverse effects on humans can be removed or reduced, and thus the operation efficiency of the air-conditioning apparatus is improved.

As described above, the temperature and treatment air amount of the air-conditioning apparatus are controlled as follows so that a limited range from a floor surface is filled with high-concentration ozone. This control is executed by, for example, the control unit 11.

With a person staying in the space, the air-conditioning apparatus is operated normally. The electrical precipitator unit 10 can be operated to the extent that the ozone concentration does not exceed the environmental standard value, and fine particles can be removed. At this time, the voltage or energization condition of the electrical precipitator unit 10 may be adjusted in accordance with an increase or decrease in ozone concentration by measuring the ozone concentration at all times.

Basically, in a time slot when no one stays in the space, deodorization or sterilization work is prepared first. This is work for being capable of effectively supplying cold air to the lower part of the space in the next stage of deodorization or sterilization work. In this preparatory stage, the supply air amount is set to, for example, the maximum. As a result, the indoor environment is settled in a short time. The temperature of air supplied by the FCU is set high and the humidity is also set high. Examples of the setting include 50% or more in relative humidity at 28° C. At this time, the electrical precipitator unit 10 is turned OFF or is operated to the same extent as in the normal operation. It should be noted that the humidity is raised so that sterilization with ozone is more effectively performed as it is generally known that viruses are difficult to survive in high-humidity conditions.

Next, deodorization or sterilization work is performed. The treatment air amount is reduced in this stage. Then, the temperature of air supplied by the FCU is set low. For example, the temperature is set to be approximately 3 degrees or more lower than the indoor environment. The humidity control is turned OFF. As a result, the ozone concentration is maintained. Then, the electric power of the electrical precipitator unit 10 is set to the maximum and the ozone generation amount is increased. It is desirable to adjust the indoor ozone concentration to 0.1 ppm or more and 0.25 ppm or less. It should be noted that with a safety measure taken regarding the entry of people, the concentration described above can be further increased and sterilization can be effectively performed in a short time. As a result of the treatment air amount, temperature, and humidity setting and operation of the electrical precipitator unit 10, ozone concentration-raised high-density cold air is supplied to the lower part of the space. At this time, mixing and stirring with the indoor air are suppressed, filling with the cold air occurs slowly and gradually from the place near the floor surface, and the cold air is introduced into the entire lower part of the space. Then, the part can be intensively deodorized or sterilized by allowing the high-ozone concentration air to stand still at the lower part of the space.

In a case where the deodorization or sterilization work is completed, an operation for reducing the ozone concentration in the space is performed. For example, outside air is introduced into the space. Alternatively, the electrical precipitator unit 10 is turned OFF or operated to the same extent as in the normal operation while increasing the treatment air amount of the air-conditioning apparatus. The ozone concentration is reduced by actively mixing and stirring the indoor air. A person can stay in the space after a state where the ozone concentration has reliably decreased is confirmed. Although normal operation initiation immediately after the end of an unmanned time slot is illustrated in FIG. 11, the normal operation initiation may precede the end of the unmanned time slot.

It should be noted that the generated ozone may be adsorbed onto the medium-efficiency particulate air filter 33. In order to desorb the adsorbed ozone from the medium-efficiency particulate air filter 33, a period in which the operation of the electrical precipitator unit 10 is turned OFF is provided. For example, ozone desorption is performed by operating only a fan while turning OFF the electrical precipitator unit 10 in an unmanned state or in a time slot when a high level of dust removal is not required. In a manned normal operation time slot of the air-conditioning apparatus, ozone generated at the electrical precipitator unit 10 is adsorbed on the filter, and thus the ozone concentration in the space gradually rises, and the ozone concentration becomes constant at ozone adsorption saturation.

The air-conditioning apparatus described in each embodiment described above is, for example, grasped as follows.

An air-conditioning apparatus (1) according to the present disclosure includes: an electrical precipitator unit (10) including a discharge electrode (31) having a main body portion (31A) and a corona discharge portion (31B, 31C) for corona discharge protruding from the main body portion and a collecting electrode (32) installed to face the discharge electrode; and a medium-efficiency particulate air filter unit (12) installed downstream of the electrical precipitator unit, in which the collecting electrode is a plate-shaped member and has a plate surface provided parallel to a gas flow direction, and the corona discharge portion has a first corona discharge portion (31B) protruding from the main body portion toward an upstream side in the gas flow direction in one side end portion of the main body portion and a second corona discharge portion (31C) protruding from the main body portion toward a downstream side in the gas flow direction in the other side end portion of the main body portion.

According to this configuration, the electrical precipitator unit is provided with the discharge electrode and the collecting electrode, corona discharge occurs by voltage application to the discharge electrode, and dust (particulate matter) energized as a result of the corona discharge is collected on the collecting electrode. The collecting electrode, which is a plate-shaped member, has a plate surface provided parallel to the gas flow direction, and gas flows between the discharge electrode and the collecting electrode. The first corona discharge portion protrudes from the main body portion toward the upstream side in the gas flow direction in one side end portion of the main body portion of the discharge electrode, and the second corona discharge portion protrudes from the main body portion toward the downstream side in the gas flow direction in the other side end portion of the main body portion of the discharge electrode.

The discharge electrode can be subjected to corona discharge from the corona discharge portion toward the collecting electrode to allow ionic wind to flow. In addition, collection performance improvement is achieved since the corona discharge portions are provided in a plurality of stages.

Further, dust in the gas is collected by the medium-efficiency particulate air filter unit. With the medium-efficiency particulate air filter unit, pressure loss and replacement frequency can be reduced. In addition, since the electrical precipitator unit is provided with the plurality of stages of corona discharge portions, a sufficient amount of energization can be applied to particles, a strong electrostatic force acts on the medium-efficiency particulate air filter unit, and thus the collection performance is improved.

In the air-conditioning apparatus according to the above disclosure, a negative charge may be applied to the discharge electrode.

According to this configuration, a negative charge is applied to the discharge electrode, stable discharge is possible, and ozone is generated with ease at the time of discharge.

The air-conditioning apparatus according to the above disclosure may further include: an ozone concentration measuring unit (40) installed in a space supplied with air that has passed through the electrical precipitator unit and the medium-efficiency particulate air filter unit and measuring ozone concentration in the space; and a control unit (11) adjusting voltage or energization condition applied to the discharge electrode of the electrical precipitator unit based on the measured ozone concentration.

According to this configuration, the ozone concentration measuring unit is installed in the space, air is supplied to the space after passage through the electrical precipitator unit and the medium-efficiency particulate air filter unit, the ozone concentration measuring unit measures the ozone concentration in the space, and the voltage or energization condition applied to the discharge electrode of the electrical precipitator unit is adjusted based on the measured ozone concentration. As a result, the amount of ozone generated as a result of the corona discharge at the discharge electrode is adjusted, and thus the ozone concentration in the space can be increased or decreased.

The air-conditioning apparatus according to the above disclosure may further include an environmental condition measuring unit installed in a space supplied with air that has passed through the electrical precipitator unit and the medium-efficiency particulate air filter unit and measuring an environmental condition in the space, in which the control unit may adjust the voltage or the energization condition applied to the discharge electrode of the electrical precipitator unit based on the measured ozone concentration and the measured environmental condition.

According to this configuration, the environmental condition measuring unit is installed in the space, air is supplied to the space after passage through the electrical precipitator unit and the medium-efficiency particulate air filter unit, and the environmental condition measuring unit measures the environmental conditions in the space such as temperature and humidity. Then, the voltage applied to the discharge electrode of the electrical precipitator unit is adjusted based on the measured ozone concentration and the measured environmental conditions. As a result, the amount of ozone generated as a result of the corona discharge at the discharge electrode is adjusted in view of the environmental conditions as well as the ozone concentration measured by the ozone concentration measuring unit, and thus the ozone concentration in the space can be increased or decreased with high accuracy so as to match the actual ozone concentration.

The air-conditioning apparatus according to the above disclosure may further include: an environmental condition measuring unit installed in a space supplied with air that has passed through the electrical precipitator unit and the medium-efficiency particulate air filter unit and measuring an environmental condition in the space; and a control unit adjusting voltage or energization condition applied to the discharge electrode of the electrical precipitator unit based on a relationship between the voltage applied to the discharge electrode and the measured environmental condition.

According to this configuration, the environmental condition measuring unit is installed in the space, air is supplied to the space after passage through the electrical precipitator unit and the medium-efficiency particulate air filter unit, and the environmental condition measuring unit measures the environmental conditions in the space such as temperature and humidity. Then, the voltage applied to the discharge electrode of the electrical precipitator unit is adjusted based on the relationship between the voltage applied to the discharge electrode and the measured environmental condition. As a result, the amount of ozone generated as a result of the corona discharge at the discharge electrode is adjusted, and thus the ozone concentration in the space can be increased or decreased.

In the air-conditioning apparatus according to the above disclosure, the control unit may switch between a first mode in which the voltage or energization condition applied to the discharge electrode of the electrical precipitator unit is adjusted such that the measured ozone concentration becomes equal to or greater than a predetermined threshold value and a second mode in which the voltage or energization condition applied to the discharge electrode of the electrical precipitator unit is adjusted such that the measured ozone concentration becomes less than the threshold value.

According to this configuration, in the first mode, the voltage applied to the discharge electrode of the electrical precipitator unit is adjusted such that the measured ozone concentration becomes equal to or greater than a predetermined threshold value and, in the second mode, the voltage applied to the discharge electrode of the electrical precipitator unit is adjusted such that the measured ozone concentration becomes less than the predetermined threshold value, and switching occurs between the first mode and the second mode. For example, in the first mode, the ozone concentration is increased such that the space supplied with air is forcibly deodorized or sterilized by ozone and, in the second mode, the ozone concentration is lowered to the extent that ozone does not adversely affect a person staying in the space.

In the air-conditioning apparatus according to the above disclosure, the adjustment of the energization condition may be a change in energizing rate in an intermittent energization condition.

According to this configuration, the energization condition is adjusted and the ozone concentration is changed by changing the energizing rate in the intermittent energization condition.

In the air-conditioning apparatus according to the above disclosure, the control unit may supply air relatively high in temperature to a space and then supply air relatively small in air amount and relatively low in temperature to the space and may adjust the voltage or energization condition applied to the discharge electrode of the electrical precipitator unit such that the measured ozone concentration becomes equal to or greater than a predetermined threshold value.

According to this configuration, the space is supplied with air relatively high in temperature and then air relatively small in air amount and relatively low in temperature. At this time, control is performed such that the voltage or energization condition applied to the discharge electrode of the electrical precipitator unit is adjusted and the measured ozone concentration becomes equal to or greater than a predetermined threshold value. As a result, mixing and stirring with indoor air is suppressed, filling with cold air occurs slowly and gradually from a place near a floor surface, and the cold air is introduced into the entire lower part of the space. Then, the lower part of the space can be intensively deodorized or sterilized.

### REFERENCE SIGNS LIST

1: air-conditioning apparatus

2: outside air treatment air conditioner (external air conditioner)

3: air handling unit (AHU)

4: duct

5: duct

6: duct

7: damper

8: damper

9: casing

10: electrical precipitator unit

11: control unit

12: medium-efficiency particulate air filter unit

13: air conditioning unit

14: washing liquid supply unit

15: supply pipe

16: valve

17: recirculation pipe

18: drain pipe

19: casing

31: discharge electrode

31A: main body portion

31B: corona discharge portion (first corona discharge portion)

31C: corona discharge portion (second corona discharge portion)

32: collecting electrode

33: medium-efficiency particulate air filter

40: ozone concentration measuring unit

50, 50A, 50B, 50C: space

1. An air-conditioning apparatus comprising:
an electrical precipitator unit including a discharge electrode having a main body portion and a corona discharge portion for corona discharge protruding from the main body portion and a collecting electrode installed to face the discharge electrode; and
a medium-efficiency particulate air filter unit installed downstream of the electrical precipitator unit, wherein
the collecting electrode is a plate-shaped member and has a plate surface provided parallel to a gas flow direction, and
the corona discharge portion has a first corona discharge portion protruding from the main body portion toward an upstream side in the gas flow direction in one side end portion of the main body portion and a second corona discharge portion protruding from the main body portion toward a downstream side in the gas flow direction in the other side end portion of the main body portion.

2. The air-conditioning apparatus according to claim 1, wherein a negative charge is applied to the discharge electrode.

3. The air-conditioning apparatus according to claim 1, further comprising:
an ozone concentration measuring unit installed in a space supplied with air that has passed through the electrical precipitator unit and the medium-efficiency particulate air filter unit and measuring ozone concentration in the space; and
a control unit adjusting voltage or energization condition applied to the discharge electrode of the electrical precipitator unit based on the measured ozone concentration.

4. The air-conditioning apparatus according to claim 3, further comprising an environmental condition measuring unit installed in the space supplied with air that has passed through the electrical precipitator unit and the medium-efficiency particulate air filter unit and measuring an environmental condition in the space, wherein
the control unit adjusts the voltage or the energization condition applied to the discharge electrode of the electrical precipitator unit based on the measured ozone concentration and the measured environmental condition.

5. The air-conditioning apparatus according to claim 1, further comprising:
an environmental condition measuring unit installed in a space supplied with air that has passed through the electrical precipitator unit and the medium-efficiency particulate air filter unit and measuring an environmental condition in the space; and
a control unit adjusting voltage or energization condition applied to the discharge electrode of the electrical precipitator unit based on a relationship between the voltage applied to the discharge electrode and the measured environmental condition.

6. The air-conditioning apparatus according to claim 3, wherein the control unit switches between a first mode in which the voltage or energization condition applied to the discharge electrode of the electrical precipitator unit is adjusted such that the measured ozone concentration becomes equal to or greater than a predetermined threshold value and a second mode in which the voltage or energization condition applied to the discharge electrode of the electrical precipitator unit is adjusted such that the measured ozone concentration becomes less than the threshold value.

7. The air-conditioning apparatus according to claim 3, wherein an adjustment of the energization condition is a change in energizing rate in an intermittent energization condition.

8. The air-conditioning apparatus according to claim 3, wherein the control unit supplies air relatively high in temperature to the space and then supplies air relatively small in air amount and relatively low in temperature to the space and adjusts the voltage or energization condition applied to the discharge electrode of the electrical precipitator unit such that the measured ozone concentration becomes equal to or greater than a predetermined threshold value.
